(19) United States
(12) Patent Application Publication
(10) Pub. No.: US 2022/0354854 A1
(43) Pub. Date: Nov. 10, 2022

(54) Treatment of Conditions Associated With Decreased Folate in Cerebral Spinal Fluid

(71) Applicant: Linzy O. Scott, III, Atlanta, GA (US)

(72) Inventor: Linzy O. Scott, III, Atlanta, GA (US)

(21) Appl. No.: 17/871,314

(22) Filed: Jul. 22, 2022

Related U.S. Application Data
(63) Continuation of application No. 17/546,654, filed on Dec. 9, 2021, now abandoned.
Application No. 17/546,654 is a continuation of application No. 17/243,902, filed on Apr. 29, 2021, now abandoned.
Application No. 17/243,902 is a continuation of application No. 17/025,538, filed on Sep. 18, 2020, now abandoned.
Application No. 17/025,538 is a continuation of application No. 16/778,580, filed on Jan. 31, 2020, now abandoned.
Application No. 16/778,580 is a continuation of application No. 16/449,178, filed on Jun. 21, 2019, now abandoned.
Application No. 16/449,178 is a continuation of application No. 16/173,122, filed on Oct. 29, 2018, now abandoned.
Application No. 16/173,122 is a continuation of application No. 15/909,647, filed on Mar. 1, 2018, now abandoned.
Application No. 15/909,647 is a continuation of application No. 15/656,630, filed on Jul. 21, 2017, now abandoned.
Application No. 15/656,630 is a continuation of application No. 15/374,002, filed on Dec. 9, 2016, now abandoned.
Application No. 15/374,002 is a continuation of application No. 15/137,443, filed on Apr. 25, 2016, now abandoned.
Application No. 15/137,443 is a continuation of application No. 14/519,898, filed on Oct. 21, 2014, now abandoned.
Application No. 14/519,898 is a continuation of application No. 14/193,627, filed on Feb. 28, 2014, now abandoned.
Application No. 14/193,627 is a continuation of application No. 13/934,671, filed on Jul. 3, 2013, now abandoned.
Application No. 13/934,671 is a continuation of application No. 13/347,622, filed on Jan. 10, 2012, now abandoned.
(60) Provisional application No. 61/460,856, filed on Jan. 10, 2011.

(51) Int. Cl. A61K 31/519; A61K 31/205; A61K 33/06; A61K 31/59; A61K 31/714; A61K 45/06; A61K 33/26

(52) U.S. Cl. CPC A61K 31/519; A61K 31/205; A61K 33/06; A61K 31/59; A61K 31/714; A61K 45/06; A61K 33/26

(57) ABSTRACT

The present invention provides methods and compositions for treating endocrine conditions that result in decreased folate levels in the cerebrospinal fluid. These conditions may be prevented and treated with reduced folates and vitamin B12. Administration of reduced folates and vitamin B12 will prevent or treat cerebrospinal folate deficiency, which is linked to thyroid-related medical conditions. Administration of reduced folates and vitamin B12 will also prevent or treat conditions associated with masked megaloblastic anemia and hypothyroidism, and other conditions brought upon through improper thyroid function. Methods of diagnosing decreased folate levels in individuals are also disclosed.

### CROSS REFERENCE TO RELATED APPLICATIONS

This application claims the benefit of priority to U.S. Provisional Application No. 61/460,856, filed on Jan. 10, 2011, which is incorporated by reference herein in its entirety.

### FIELD

The subject matter disclosed herein generally relates to methods of diagnosing, treating, and preventing conditions associated with decreased folate in cerebral spinal fluid.

### BACKGROUND

Folate is a critical vitamin that is required for proper nutrition. Folate is important in forming DNA and RNA; therefore, it is critical in cells that are growing or undergo frequent cell division. Folate deficiencies have led to harmful and serious health conditions in children as well as in adults. As a result, folate is especially important for pregnant mothers, nursing mothers and newborns.

Neural tube defects in fetuses are perhaps the most common problem associated with folate deficiency. Expecting mothers are routinely placed on a folic acid regimen. Additionally, nursing mothers are also supplemented with folic acid to continue to provide nutrition to the newborn. During the prenatal and perinatal periods, folate is essential for adequate enclosure of the neural tube by dermal tissues. In recent studies, it has been shown that women with increased levels of plasma homocysteine and decreased levels of erythrocyte folate have a greater risk of having an offspring with a neural tube defect. It is believed that during the early stages of pregnancy (prior to the development of the placenta) transport of folates to the fetus is primarily performed by the maternal erythrocytes. Inadequate folate levels in maternal erythrocytes are a significant factor in the lack of progression of neural tube closure in utero.

Folate helps produce and maintain new cells; this is critically important in cells with rapid growth that undergo frequent cell division such as in infancy and pregnancy. Folate is needed to form DNA and RNA, and both adults and children need folate to make normal red blood cells. Folates also play a critical role in the reduction of plasma homocysteine levels. An increased amount of homocysteine in the plasma has been associated with heart disease. Folates have been shown to reduce the calcification of plaques during an acute ischemic attack; thereby reducing the long-term effects of cardiovascular disease. Thus, folates are major components of cardiovascular functionality.

Endocrine conditions (including conditions affecting the thyroid), and medications that are used in connection with endocrine conditions, are known to cause hematological issues in individuals, as well as in (i) fetuses of such individuals who may be pregnant, or (ii) children who receive breast milk from such individuals who have endocrine issues and/or who are on endocrine medication. In addition, these endocrine conditions, and the medications that are used in connection with endocrine conditions, are known to cause (a) adverse hepatic conditions with respect to the liver, as well as adverse impacts on other organs, (b) adverse mitochondrial conditions, and (c) adverse oxidative phosphorylation, adenosine triphosphate or other oxidative issues. Moreover, in addition to those individuals who have endocrine conditions or who are taking medications for endocrine conditions, environmental conditions and environmental contaminants are also known to impact the endocrine system, the hepatic system, other vital organs, hematology, mitochondria and oxidative processes of an individual, as well as the fetus of such individual or child nursing from such individual. Such foregoing environmental conditions and environmental contaminants include, but are not limited to, toxins used in or emitted from manufacturing, welding, energy production, pesticides, fertilizers and water treatment, and those relating to radiation. Thus, the endocrine system (as well as other facets of the human body as addressed above) can be impacted by (a) developments within one's own body (for example, autoimmune complications leading to thyroid issues), (b) certain medications, and (c) the environment or environmental contaminants.

Typically, endocrine conditions, and in this instance, specifically the thyroid, are treated with medication to address the thyroid condition and bring the patient to a euthyroid state. That is the focus of the medical community and pharmaceutical community. However, the medical and pharmaceutical communities do not sufficiently address the further complications the endocrine conditions, and/or the medications that are used in connection with the endocrine conditions, cause. In addition, the medical and pharmaceutical communities (as well as the communities or industries that deal with environmental toxins) do not sufficiently address other conditions that affect the hepatic system and hematology. For instance, antithyroid drugs (as well as a number of toxins, including, but not limited to, aluminum, arsenic, benzene, beryllium, cadmium, carbon monoxide, chromium, copper, iron, manganese, nickel, nitric oxide, silver, zinc, and radiation) are known to cause (i) numerous blood disorders (megaloblastic anemia, pancytopenia, aplastic anemia, neutropenia, agranulocytosis, thrombocytopenia or leukopenia, among others), (ii) bone marrow suppression, and (iii) hepatic dysfunction. Further, endocrine conditions are known to cause similar conditions. For example, hypothyroidism is known to cause iron, folate and vitamin B12 anemias, which with respect to folate or vitamin B12 anemias, can cause “macrocytic” or “megaloblasitc” hematological conditions leading to bone marrow suppression and hepatic dysfunction, as well as dysfunction in other organs (polyglandular issues for instance). Hypothyroidism has also been associated with adverse mitochondrial outcomes and oxidative processes. Even further, autoimmune conditions like chronic autoimmune thyroiditis and Hashimoto's Thyroiditis compounded by pernicious anemia can cause even further vitamin B12 deficiencies that will not be corrected solely by thyroid hormone replacement, but also require specific vitamin B12 supplementation. Moreover, what further complicates the clinical picture with respect to endocrine conditions as well as other conditions affecting cerebral folate, is (1) “masked megaloblastic anemia” that can arise from simultaneous iron and folate/vitamin B12 deficiencies, as well as other microcytic/normocytic/macrocytic anemias, (2) “masked” neutropenia or agranulocytosis that can arise from certain physiological processes in newborns, (3) lack of vitamin B12 which is critical in the pathway of converting folate into the form that is needed in cerebrospinal fluid (i.e., one could have folate, but lack the vitamin B12 necessary to convert the folate to the form needed in the brain thereby leading to cerebral folate deficiency; vitamin B12 also assists in the metabolic process of S-adenosylmethionine which metabolic pathway is also critical for neurological function), and (4) “polymorphisms” that are commonplace within our populations. For instance, the methylenetetrahydrofolate/methyltetrahydrofolate (MTHFR) (C667T or A1298C) polymorphism is very common, by some accounts up to 40% of the population. In addition, some individuals have polymorphisms related to DIO2 or OATP1c1. That means some individuals are naturally more susceptible to having cerebral folate issues, or ancillary folate and/or vitamin B12 issues, than others based on whether or not they have the polymorphism. Yet, notwithstanding the foregoing, the medical and pharmaceutical communities (as well as the communities and industries that deal with environmental toxins) do not do enough to address these complications, and as a result, populations are suffering from cerebral folate deficiency or ancillary folate and/or vitamin B12 deficiencies.

The disclosed subject matter is based on the discovery that these various conditions associated with endocrine conditions can create decreased folate levels in cerebrospinal fluid, which can be avoided by monitoring folate and vitamin B12 levels, and supplementing with folate, vitamin B12 or folinic acid/reduced folates as necessary. Since folate and vitamin B12 are part of the “Vitamin B Complex” and the homeostasis of the Vitamin B Complex can be affected by fluctuations in folate and/or vitamin B12 levels and vice versa, the disclosed subject matter also addresses and includes the Vitamin B Complex as a whole. Further, certain amino and organic acids, as well as cofactors, enzymes and compounds (collectively, “Other Elements”) are referenced herein as well, and the disclosed subject matter also addresses and includes such elements. Moreover, the subject matter disclosed herein includes 5-methyltetrahydrofolate levels specifically in the brain/cerebrospinal fluid, (i) derivatives of methyltetrahydrofolate, including methylenetetrahydrofolate and 5,10-methylenetetrahydrofolate in the brain/cerebrospinal fluid, and (ii) derivatives of folate, folic acid, folinic acid, other vitamins of the Vitamin B Complex, and the amino and organic acids, cofactors, enzymes and compounds referenced herein (including with respect to (i) and (ii), new biochemical or natural versions of such vitamins or methyl versions and means of delivery of such vitamins or methyl versions).

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to therapies for treating conditions associated with decreased folate levels in the cerebrospinal fluid. Such conditions can be caused by, for example, masked megaloblastic anemia, endrocrine conditions (e.g., hypo or hyperthyroid conditions), environmental causes, and the like. Still further, the disclosed methods relate to methods of diagnosing decreased folate levels in the cerebrospinal fluid in various types of individuals.

In specific examples, the disclosed subject matter relates to methods and compositions for preventing and/or treating people with thyroid-related medical conditions from developing problems associated with folate deficiencies. In some embodiments, the disclosed subject matter provides a method of administering folate to people with endocrine conditions like thyroid-related medical conditions. In some embodiments, the disclosed subject matter provides a method of administering folate and vitamin B12 to people with thyroid-related medical conditions. In some embodiments, the disclosed subject matter further provides a method of administering a reduced folate to people with thyroid-related medical conditions. In some embodiments, the disclosed subject matter further provides a method of administering a reduced folate and vitamin B12 to people with thyroid-related medical conditions. Yet in another embodiment, the disclosed subject matter further provides a method of administering folinic acid and vitamin B12 to people with thyroid-related medical conditions. And in some embodiments the administration of folate and vitamin B12 will treat or prevent cerebrospinal folate deficiency, masked megaloblastic anemia, other macrocytic anemias (which include anemias that may be masked macrocytic anemias), or hepatic dysfunction. In some embodiments, the disclosed subject matter includes the administration of folate and vitamin B12 and can be coupled with the administration of iron. Other embodiments include the administration of L-carnitine and/or calcium and/or vitamin D along with the administration of folate and vitamin B12 to individuals in need thereof. With respect to calcium and vitamin D, these are preferred embodiments that also address parathyroid hormone deficiencies. Other embodiments include the administration of certain vitamins of the Vitamin B Complex and/or Other Elements coupled with folate or vitamin B12.

In some embodiments, the disclosed subject matter provides a method of administering folate and vitamin B12 to people with hypothyroidism or hyperthyroidism. In other embodiments, the disclosed subject matter provides a method of administering folate and vitamin B12 to people that have been treated with radioactive iodine, or who have had surgery on or related to their thyroid, or who have had any procedure that has reduced the size or activity of their thyroid gland. In another embodiment, the disclosed subject matter provides a method of administering folate and vitamin B12 to an individual having hypothyroxinemia or another temporary period of hypothyroidism. In yet another embodiment, the disclosed subject matter provides a method of administering folate and vitamin B12 to an individual that is a fetus or nursing child of a mother or caregiver who has a thyroid-related medical condition.

In some embodiments, the disclosed subject matter provides a composition of a thyroid stimulating drug, a folate, and vitamin B12. This embodiment facilitates prevention and treatment of folate deficiencies for persons that have hypothyroidism. In other embodiments, the compositions additionally include iron, and/or L-carnitine, and/or calcium, and/or vitamin D. In other embodiments, the compositions additionally include certain vitamins of the Vitamin B Complex and/or Other Elements. In another embodiment, the disclosed subject matter provides a composition of an anti-thyroid drug, a folate, and vitamin B12. This embodiment facilitates prevention and treatment of folate deficiencies for persons that are being treated for hyperthyroidism and can also be complemented by iron, and/or L-carnitine, and/or calcium, and/or vitamin D, as well as certain vitamins of the Vitamin B Complex and/or Other Elements.

In a preferred embodiment, the methods and compositions for prevention and treatment of thyroid-related medical conditions comprise 5-methyltetrahydrofolic acid, or another reduced folate, and vitamin B12. In another preferred embodiment, the composition for prevention and treatment of thyroid-related medical conditions comprise 5-methyltetrahydrofolic acid, or another reduced folate, and vitamin B 12 with either an anti-thyroid drug or a thyroid stimulating drug. In another preferred embodiment, the composition of anti-thyroid drug or thyroid stimulating drug, folate or another reduced folate, and vitamin B 12 also comprise iron, and/or L-carnitine, and/or calcium, and/or vitamin D, as well as certain vitamins of the Vitamin B Complex and/or Other Elements.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

The materials, compounds, compositions, and methods described herein may be understood more readily by reference to the following detailed description of specific aspects of the disclosed subject matter, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a.” “an,” and “the” include plural referents unless the context clearly dictates otherwise.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about.” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “prevent” or other forms of the word, such as “preventing” or “preventative” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as neurological damage), diminishment of extent of neurological damage, or stabilized (i.e., not worsening) state of neurological damage.

The term “individual” preferably refers to a human in need of treatment with a composition as disclosed herein to treat decreased cerebral spinal folate levels. However, the term “individual” can also refer to non-human animals, preferably mammals such as dogs, cats, horses, cows, pigs, sheep and non-human primates, among others, that are in need of treatment with the compositions disclosed herein.

As used herein, “folate(s)” are a group of pteroylglutamate acids that become structurally and functionally altered when reduced. The term “folate” refers to folic acid and any derivatives thereof. Folic acid, (N-[4-(2-Amino-3,4-dihydro-4-oxo-6-pteridinylmethylamino)-benzoyl]-L-glutamic acid) also known as vitamin B9 or folicin as well as N-pteroyl-L-glutamic acid and N-pteroyl-L-glutamate, is a non-reduced folate. In humans, folates are absorbed most readily as the most active form 6(R,S)-5-methyltetrahydrofolate (6(S)-5-methyltetrahydrofolate being the most biologically active) and it is the principal circulating form of folate (referred to herein as “reduced folate”). A nonexclusive list of other reduced folates (also included in the definition of “reduced folates”) are 10-methylenetetrahydrofolate, 10-formyltetrahydrofolic acid, 5-formyltetrahydrofolic acid, 5-forminino tetrahydrofolic acid, 5,10-methenyltetrahydrofolic acid, 5,10-methyltetrahydrofolic acid, L-methylfolate, and 6(R,S)-5-formyltetrahydrofolate (folinic acid), and tetrahydrofolic acid/tetrahydrofolate. The term “folate” is used as a genus, and generally refers to any of these forms of folate: folic acid, any form of reduced folates, and 5-methyltetrahydrofolic acid. It also can include references to Isovorin, Wellcovorin, Leucovorin and Metafolin.

By “Vitamin B Complex” is meant any one or more of the following compounds, vitamin B1 (thiamine), Vitamin B2 (riboflavin), Vitamin B3 (niacin or niacinamide). Vitamin B5 (pantothenic acid), Vitamin B6 (pyridoxine, pyridoxal, or, pyridoxamine, or pyridoxine hydrochloride), Vitamin B7 (biotin), Vitamin B9 (folic acid), Vitamin B12.

Vitamin B12, also called cobalamin, is a water soluble vitamin. Vitamin B12 refers to a group of cobalt-containing vitamer compounds known as cobalamins: these include cyanocobalamin, hydroxocobalamin, and the two naturally occurring cofactor forms of Bi2 in the human body: δ′-deoxvadenosylcobalamin (adenosylcobalamin—AdoB12), the cofactor of Methylmalonyl Coenzyme A mutase (MUT), and methylcobalamin (MeBi2), the cofactor of 5-methyltetrahydrofolate-homocysteine methyltransferase (MTR).

By the term “Other Elements” is meant one or more of the following compounds, calcium, aconitic, alanine, alpha amino butyric acid, arginine, cystine, ethanolamine, glutamate, glutamic acid, glutamine, glutaric, glycine, histidine, homocysteine, hydroxyproline, isoleucine, lactate, lactic acid, lysine, methionine, 1-methylhistidine, 3-methylhistidine, phenylalanine, serine, threonine, tryptophan, tyrosine valine, and/or L-carnitine, iron, and vitamin D, including metabolic precursors of these.

The term “cerebrospinal folate deficiency” (also referred to as cerebral folate deficiency) is associated with decreased levels of 5-methyltetrahydrofolate in the cerebrospinal fluid (CSF). In some conditions, the decreased level of folate in CSF is also associated with normal folate levels in the plasma and red blood cells. The onset of symptoms caused by the deficiency of folates in the brain generally begin within the first year of life, but in the examples contained herein exhibited themselves at birth or within the immediate months thereafter. This is followed by delayed development, with deceleration of head growth, hypotonia, and ataxia, followed in many cases by dyskinesias (choreo-athetosis, hemiballismus), spasticity, and speech difficulties, as well as numerous other cognitive, social, behavioral, psychological and physical conditions.

The term “masked megaloblastic anemia” is characterized by folate and/or vitamin B12 deficiencies occurring simultaneously with an iron deficiency, such that the iron deficiency masks the red blood cell indices changes of megaloblastic anemia.

The term “masked macrocytic anemias” refers to conditions where a macrocytic anemia is masked, and includes (a) masked megaloblastic anemia, (b) when a macrocytic anemia is masked by a microcytic to normocytic anemia that occurs simultaneously with the macrocytic anemia, or (c) neutropenia that is masked at birth, in part, by a phenomena whereby neutrophil counts and white blood cell values rise immediately after birth.

The term “hypothyroxinemia” refers to conditions associated with the presence of an abnormally low concentration of thyroxine in the blood.

The term “iron” as it relates to nutritional supplementation, refers to any form of iron that is generally known to supplement nutrition; for example, an iron (II) salt, an iron (III) salt, or carbonyl iron.

The term “anti-thyroid drug” is a drug, agent or medication directed against the thyroid gland for the purposes of reducing thyroid function. The anti-thyroid drugs include, but are not limited to, carbimazole, methimazole, potassium perchlorate, potassium iodide and propylthiouracil (PTU). These drugs are used to treat hyperthyroidism (overactivity of the thyroid gland) or other thyroid-related medical conditions, primarily in order to reduce the excessive thyroid activity before surgery and to treat and maintain patients not having surgery.

The term “thyroid stimulating drug” is a drug, agent, medication or hormone that acts as a replacement for a hormone that is normally produced by the thyroid gland to regulate the body's energy and metabolism. These drugs are used for the purpose of increasing thyroid function. Thyroid stimulating drugs include but are not limited to. Levothyroxine, Levothyroxine Sodium. Liothyronine Sodium, Liotrix, Thyroglobulin, Thyroid, Thyroxine, Triiodothyronine, Levoxyl, Synthroid, Levo-T, Unithroid, Levothroid, Levoxine, Levolet, Novothyrox, Triostat, Cytomel and Thyrolar.

The term “thyroid-related medical condition” refers to medical conditions that arise when the thyroid gland is not functioning properly. This could include hypothyroidism (under active thyroid function), hyperthyroidism (overactive thyroid function), anatomical disorders, and tumors (including thyroid cancer). “Thyroid-related medical conditions” also arise from and include the use of agents, drugs or medications to treat the thyroid, or from environmental toxins or environmental conditions that impact the thyroid. The term “thyroid-related medical conditions” also includes complications associated with, diabetes, diabetes mellitus, hypoparathyroidism and polyglandular failure syndrome brought about in connection with a thyroid gland that is not functioning properly.

Reference will now be made in detail to specific aspects of the disclosed materials, compounds, compositions, and methods, examples of which are illustrated in the accompanying Examples.

### Materials and Methods

What has previously been unknown is the relationship between the thyroid and levels of folate, as well as vitamin B12, in the blood as they relate to cerebrospinal folate and the adverse affects of having a thyroid condition. Hypothyroid individuals have been found to suffer folate, as well as, vitamin B12 deficiencies; and as such, they are prone to the other problems that are also associated with decreased folate levels. It is now discovered that conditions of hypothyroidism have led to folate deficiencies in cerebrospinal fluid. There has been a newly discovered case involving the treatment of hyperthyroidism that has also led to folate deficiencies in cerebrospinal fluid. This is because the drugs that are taken to treat hyperthyroidism suppress the thyroid and in some cases have suppressed it to the extent that it leads to hypothyroidism and folate deficiencies in cerebrospinal fluid. In addition, these anti-thyroid drugs can cause adverse hematological and hepatic conditions that can also contribute to deficiencies in folate, as well as vitamin B12, leading to cerebrospinal folate deficiency. This surprising discovery has led to the present invention.

Providing individuals, who have had or are at risk of having thyroid-related medical conditions, with folate and vitamin B12 has shown to beneficially address and alleviate adverse outcomes associated with decreased folate in cerebrospinal fluid. The present invention also addresses those who must take anti-thyroid drugs or thyroid stimulating drugs or hormones. Supplementation with folates and vitamin B12 along with either anti-thyroid drugs or thyroid stimulating drugs can provide a better means of preventing and/or treating folate deficiencies and the associated problems from such deficiencies.

This invention can help prevent and further help diagnose the cause of folate deficiencies in some individuals, as thyroid-related medical conditions are presently not part of the focus of the medical and pharmaceutical communities. Further, leading researchers in the field of cerebrospinal folate deficiency have mainly focused on antibodies attacking the folate receptor or mitochondrial defects as the cause of cerebrospinal folate deficiency.

There is clearly a need to make the relationship between thyroid function and folate deficiencies in cerebrospinal fluid known so that it may be prevented and treated. The disclosed subject matter provides methods and compositions for prevention and treatment of endocrine conditions. The invention is based on the discovery that an improperly functioning thyroid can cause harmful conditions. Some nonexclusive examples are cerebrospinal folate deficiency and masked macrocytic anemias, and hepatic dysfunction. These conditions may be prevented or treated by the administration of folate and vitamin B12. Additionally there is a certain population of individuals who are also at risk for developing conditions that may be treated with the administration of folate and vitamin B12. Some thyroid-related medical conditions such as hypothyroidism and hyperthyroidism are treated with anti-thyroid drugs or thyroid stimulating drugs. Anti-thyroid drugs can cause harmful conditions such as macrocytic blood disorders, which may be masked macrocytic anemias, as well as hepatic dysfunction, which itself may be idiosyncratic or difficult to diagnose given its unpredictability and sudden onset. The foregoing hematological and hepatic conditions can also lead to cerebrospinal folate deficiencies. As a result, the present invention includes a composition of these drugs with the addition of folate and vitamin B12.

### Patient Population

In certain examples, patients are those who have been suffering from thyroid-related medical conditions or those who are at risk of suffering thyroid-related medical conditions, which thyroid conditions or risk of thyroid conditions can be caused by a number of circumstances, including, but not limited to, biological conditions within the patient's body, agents, drugs, or medications the patient has been exposed to, or environmental exposure to toxins, or other adverse environmental conditions.

In one embodiment, the individual with a thyroid-related medical condition may suffer from hypothyroidism or hyperthyroidism. In general, hypothyroidism is a condition in which the thyroid gland does not produce enough thyroid hormone. In general, hyperthyroidism is a condition in which the thyroid gland produces too much thyroid hormone. In a preferred embodiment, the patient is taking an anti-thyroid drug or a thyroid stimulating drug. While these types of patients may be at the highest risk, other similar conditions pose a risk that may be treated by the methods and compositions of this invention. For example, those persons with a thyroid-related medical condition and suffering from a macrocytic blood condition, masked megaloblastic anemia, masked macrocytic anemia or hepatic dysfunction, and those persons exposed to agents, drugs, medications, toxins and environmental conditions that cause any of the foregoing hematological or hepatic conditions may be treated with the methods and compositions of this invention. In another embodiment, the patient has a thyroid-related medical condition related to hypothyroxinemia. In another embodiment, the patient may be any individual treated with radioactive iodine, or who has surgery on or related to the thyroid gland, or who undergoes any other process or procedure that alters the normal function of the thyroid. In another embodiment, the patient may be a fetus or newborn with a mother or caregiver who has a thyroid-related medical condition.

### Detecting Conditions

Thyroid

One of the key discoveries of this invention is the discovery that thyroid-related medical conditions can cause cerebrospinal folate deficiencies, and the person with the thyroid condition is susceptible to all of the harms associated with cerebrospinal folate deficiencies. In one embodiment, this invention treats persons with thyroid-related medical conditions.

Hypothyroidism

In another embodiment, this invention treats persons with hypothyroidism. Hypothyroidism, or an improperly functioning thyroid, specifically not producing enough thyroid hormones, can lead to a person having cerebrospinal folate deficiencies. One of the aims of this invention is to treat people with hypothyroidism.

Hyperthyroidism

While it has been discovered that cerebrospinal folate deficiencies are more commonly associated with hypothyroidism, persons with hyperthyroidism are also the subject of this invention because they take anti-thyroid drugs to treat their hyperthyroid conditions. These drugs have the potential to lower the production of the thyroid to levels in which folate deficiencies may occur or to cause adverse macrocytic hematological or adverse hepatic conditions leading to cerebrospinal folate deficiencies. Therefore, hyperthyroidism is a condition relevant to this invention.

Diabetes, Hypoparathyroidism and Polyglandular Failure Syndrome

Thyroid-related medical conditions have been known to cause or contribute to diabetes, diabetes mellitus, hypoparathyroidism and polyglandular failure syndrome. Therefore, the conditions diabetes, diabetes mellitus, hypoparathyroidism and polyglandular failure syndrome brought upon by thyroid-related medical conditions are also the subject of this invention. With respect to diabetes, the disclosed subject matter can be coupled with drug treatments in connection with diabetes. For example, the compositions disclosed herein, such as folates and vitamin B12, can be combined with, or co-administered with, Metformin.

Pregnant

Those who are pregnant and suffer from thyroid-related medical conditions are also the subject of this invention, because the thyroid conditions a pregnant mother has can cause complications for the mother, as well as with the fetus and/or newborn.

Fetus

Because the complications of thyroid-related medical conditions may be passed from the mother to the fetus, a fetus or newborn from a mother with a thyroid-related medical condition is also the subject of this invention.

Nursing Child

Because the complications of thyroid-related medical conditions may be passed through the milk of a nursing mother to the newborn, a newborn from a mother with a thyroid-related medical condition is also the subject of this invention.

Hypothyroxinemia

Complications arising from thyroid-related medical conditions may also arise temporarily when a person is suffering from hypothyroxinemia. Periods of hypothyroxinemia have occurred during pregnancy in the mother or in the fetus. Even though this may be only a temporary period in which the thyroid is not properly functioning, harmful results may arise during this time. Therefore, hypothyroxinemia is also the subject of this invention. Further, given that hypothyroxinemia occurs in and around pregnancy and births, the disclosed compositions can also be coupled with Poly-Vi-Sol or other vitamin supplements given to neonates, infants and toddlers as a means of addressing any known, unknown or masked thyroid-related medical conditions resulting from pregnancy and birth. In a specific aspect, compositions disclosed herein such as folates and vitamin B12 can be combined with, or co-administered with, Poly-Vi-Sol or other multivitamin given to infants and neonates.

Anti-Thyroid Drugs

A person taking anti-thyroid drugs is also the subject of this invention. It has been discovered that at times taking an anti-thyroid drug can lower the function of the thyroid substantially enough to cause cerebrospinal folate deficiency, for which this invention addresses. In addition, such agent, drug, or medication also causes adverse hematological and hepatic conditions which can also lead to cerebrospinal folate deficiencies, for which this invention addresses.

Thyroid Stimulating Drugs

A person taking thyroid stimulating drugs is also the subject of this invention. As this invention addresses, hypothyroidism has been linked to cerebrospinal folate deficiency. Prior to receiving a thyroid stimulating drug, a person has for the most part already suffered from a thyroid-related medical condition. In certain cases, hypothyroidism is newly discovered in an individual and during the period in which the individual remained undiagnosed, the individual may have developed deficiencies in folate or vitamin B12 or cerebrospinal folate deficiency. In other cases, the individual may have been treated with an antithyroid drug for hyperthyroidism, and the drug caused the individual to develop hypothyroidism, and the individual then suffers from adverse events not only related to the anti-thyroid medication (the complications of which have already been addressed herein), but also the adverse conditions of having hypothyroidism. In yet another example, the individual has had hypothyroidism, but alternates between different degrees of hypothyroidism, such that the individual may be receiving at any given time an inadequate amount of thyroid stimulating drug, thereby still allowing the adverse complications of hypothyroidism to occur. In all of the foregoing instances, this invention will prevent or treat such individual.

Radioactive Iodine, Surgery, or any Other Method to Reduce the Size or Activity of the Thyroid Gland

The methods of this invention are also directed to a person who has received radioactive iodine, or who has had surgery on or related to the thyroid gland, or who has had any other procedure that has reduced the size and therefore the activity of the thyroid gland.

Hematological Conditions

It has also been discovered that macrocytic blood conditions, including masked macrocytic anemias may be brought upon by persons with thyroid-related medical conditions. As such this invention aims to prevent or treat the conditions brought upon through folate and vitamin B 12 deficiencies in persons with masked macrocytic anemias.

Hepatic Dysfunction

It has also been discovered that hepatic dysfunction may be brought upon by persons with thyroid-related medical conditions, and in some cases, the hepatic dysfunction may be idiosyncratic or difficult to diagnose given its unpredictability and sudden onset. As such, this invention aims to prevent or treat the conditions brought upon by folate and vitamin B12 deficiencies in persons with hepatic dysfunction.

### Testing Methods

In connection with the foregoing, there is also a need for diagnostic testing procedures that can either identify cerebrospinal folate deficiency or indicate a greater likelihood of being susceptible to cerebrospinal folate deficiency, thereby requiring further investigation and/or preventative measures. Currently, testing for cerebrospinal folate deficiency requires invasive procedures, such as anesthesia and/or a spinal tap. Testing of blood/plasma and/or urine provides a less invasive and less expensive procedure to identify persons suffering from, or likely to suffer from, cerebrospinal folate deficiency. References to blood/plasma means test in blood and/or plasma. Such tests can be structured as well to identify biomarkers and correlations. For instance, such tests can include testing, of blood/plasma or urine, for calcium, aconitic, hydroxyproline, lysine, lactic acid/lactate, homocysteine, glutaric, arginine, alanine, 3-methylhistidine, alpha amino butyric acid, glutamate, valine, methionine, tyrosine, tryptophan, serine, glycine, histidine, 1-methylhistidine, threonine, glutamic acid, glutamine, ethanolamine, cystine, phenylalanine and/or isoleucine, and particularly when the following correlations are found whereby “high” is indicative of values above the midpoint of the normal range and “low” is indicative of values below the midpoint of the normal range: calcium (high) range 8.8-10.1 mg/dl in blood/plasma); homocysteine (greater than <1 mmol/mol creat in urine); glutaric (greater than 5 or less mmol/mol cr in urine); aconitic (high—range 3-185 mmol/mol cr in urine); hydroxyproline (high—range 6-32 umol/L in blood/plasma); arginine (high—range 38-122 umol/L in blood/plasma); alanine (high—range 157-481 umol/L in blood/plasma; low—range 8-156 mmol/mol creat in urine); histidine (low—range 9-425 mmol/mol creat in urine); 1-methylhistidine (low—range 5-400 mmol/mol creat in urine); 3-methylhistidine (low—range 1-6 umol/L in blood/plasma; low—range 11-40 mmol/mol creat in urine); threonine (low—range 4-60 mmol/mol creat in urine); alpha amino butyric acid (high—range 6-30 umol/L in blood/plasma); lactic acid (high—range 4-16 mg/dL in blood/plasma); glutamic acid (high—range 9-109 umol/L in blood/plasma); glutamine (low—range 405-923 umol/L in blood/plasma; low—range 18-188 mmol/mol creat in urine); ethanolamine (low—range umol/L in blood/plasma; low—range 27-114 mmol/mol creat in urine); lysine (low—range 98-231 umol/L in blood/plasma; low—range 3-112 mmol/mol creat in urine); cystine (low—3-20 mmol/mol creat in urine); valine (low—range 130-307 umol/L in blood/plasma; low—range 2-20 mmol/mol creat in urine); methionine (low—range 14-37 umol/L in blood/plasma); tyrosine (low—range 31-108 umol/L in blood/plasma; low—range 3-48 mmol/mol creat in urine); tryptophan (low—range 30-94 umol/L in blood/plasma; low—range 2-27 mmol/mol creat in urine); phenylalanine (low—range 2-22 mmol/mol creat in urine); serine (low—range 85-185 umol/L in blood/plasma); glycine (high—range 138-349 umol/L in blood/plasma; low—range 23-413 mmol/mol creat in urine); and/or isoleucine (high—range 33-97 umol/L in blood/plasma).

In addition, valine/alanine substitutions can be utilized as diagnostic measures, as well as when L-carnitine values are low (range 30-89 umol/L or 20-50 umol/L or 30-60 umol/L in blood/plasma). Further, given that cerebrospinal folate deficiency ultimately impacts neurotransmitters, namely serotonin which thereby affects dopamine and other neurotransmitters, biopterin/tetrahydrobiopterin, neopterin, tyrosine and tryptophan levels in the central nervous system as well as peripheral (blood/plasma and/or urine) systems must be taken into account since they are precursors in the metabolic pathway to serotonin. It is noted that the foregoing testing factors relate to the Examples disclosed herein.

Additionally, diagnostic testing procedures can include testing for (i) polymorphisms in the MTHFR (including C677T and A1298C) or DIO2 or OATP1c1 genes. (ii) antibodies against the folate receptor, (iii) vitamin B12 (low—range 200-900 pg/mL in blood/plasma), folate (low range 3-16 ng/mL or 5-21 ng/mL in blood/plasma) or Vitamin B Complex levels, (iv) all parameters related to liver function (specifically including SGOT/AST (high—range 7-40), SGPT/ALT (low—range 37-63 U/L) and ALK PHOS (high—range 40-200 or 30-110 or 110-320 U/L) to account for idiosyncratic or sudden onset hepatic issues, (v) gastrointestinal conditions and other digestive disorders, (vi) complete blood counts (with a focus on even mildly elevated MCV, MCH and MCHC to account for masked megaloblastic anemia, as well as WBC, RBC, HGB, HCT and RDW), (vii) differentials on the complete blood count (with a focus on decreased Segmented Neutrophils, increased Lymphocytes, increased Myelocytes, increased Nucleated Red Blood Cells, increased (even if slightly) “Aniso” (anisocytosis). “Poik” (poikilocytosis), Polychrome, Hypochrome, “Macro” (macrocytes), Dohle Bodies and Toxic Granulocytes on blood smears, as well as physiological conditions leading to masked neutropenia or agranulocytosis, (viii) reticulocyte counts (with a focus on reduced RPI (reticulocyte production index), RETIC (reticulocytes) or ABS RETIC (absolute reticuloycte count)), (ix) coagulation factors, (x) the presence of jaundice, (xi) electrolyte and osmolality testing (with a focus on sodium, potassium and chloride), (xii) all parameters related to kidney function (specifically including BUN, CR and BUN/CREAT ratio (with a low ratio—range 9-21)), (xiii) phosphorus (high—range 2.0-5.0 mg/dL), albumin (low—range 2.7-4.8 or 3.5-4.8 g/dL) and magnesium levels, (xiv) C-Reactive Protein (high—range 0.0-0.8 MG/DL), (xv) the presence of cholesterol, (xvi) iron levels (low—range 11.6-35 or 4.6-30.4 or 18-45 or 9-21 umol/L), (xvii) thyroid and parathyroid levels or a diagnosis of a thyroid condition or parathyroid condition, (xviii) calcium levels, including increased calcium with hypoparathyroidism or correlation between calcium and parathyroid levels, and (xix) diabetes mellitus and other diabetic conditions (including glucose levels). It is noted that the foregoing testing factors relate to the Examples disclosed herein.

Further, with respect to testing and diagnostic procedures for the identification of “masked megaloblastic anemia,” the following correlations on complete blood counts (with differential) can be utilized whereby reference points for “low” or “high” are measured from the midpoint of the reference range; WBC (low—range 9.1-34 at birth; after birth high—range 6-14 or 3.6-11.1 10E3 uL); RBC (low—range 4.1-6.7 or 3.8-5.4 or 3.69-4.88 10E6 uL); HGB (low—range 15-24 or 10.5-14 or 11.4-14.4 g/dL); HCT (low—range 44-70 or 32-42 or 33.3-41.4%); MCV (high—range 102-115 or 72-88 or 79.3-94.8 fL); MCH (high—range 33-39 or 24-30 or 26.8-33.2 pg); MCHC (within the range of 32-36 or 33.5-35.5 g/dl); RDW (high—range 13-18 or 12-15.1%); Platelet Count (low—range 150-450 or 150-400 10E3 uL); BAND (immature neutrophils) (low—range 0-17%); SEG (segmented neutrophils (low—range 16-70%); LYMPHOCYTE (high—range 10-59 or 17-43%); MONO (monocytes) (low—range 1-23 or 5-12%); EOS (eosinophils) (low—range 0-8 or 1-8%); BASO (basophils) (low—range 0-3 or 0-1%); MYELOCYTE (high—range 0-0%); REACT LYMPH (reactive lymphocyte) (high—range 0-5%); NRBC (nucleated red blood cells (high—range 0-0 cells); GRANULOCYTE (high—range 43-72%); ANISO (slight); POLYCHROME (slight); MACRO (slight); TOXIC GRAN (toxic granulation) (slight); and DOHLE BODY (slight). It is further noted that the foregoing biomarkers are specifically well suited for infants immediately after birth, and may change due to the macro nature of blood results immediately following birth. Therefore, the foregoing correlations would have to be adjusted for infants who surpass the “macro” effects on blood due to the infant's receiving of oxygen from air rather than through the mother in gestation. Further, reticulocyte counts should be obtained with a focus on RETIC (range 3.00-7.00%), ABS RETIC (range 0.131-0.510 10E6 uL) and RPI (range 1.0-2.0), whereby such levels would be on “low”. It is noted that the foregoing testing factors relate to the Examples disclosed herein.

Further, in connection with diagnostic testing, it is noted that newborn screening is mandated throughout the United States. While the foregoing tests cover a vast array of metabolic, genetic and blood factors, many of the tests provide for thresholds that would only trigger a negative result if there is a genetic abnormality. As explained herein, subtle deficiencies over time can cause deficiencies in the Vitamin B Complex and/or cerebral folate, but may not trigger a negative result on such newborn screening. As a result, the tests identified in the immediately preceding two paragraphs can be added to metabolic screening in order that practitioners and parents can be alerted to results that can increase the likelihood of, or diagnose, deficiencies in the Vitamin B Complex or cerebral folate. Moreover, since many governmental entities may keep DNA and blood samples on record for periods of time after a child's birth, testing mechanisms to retest existing samples can be performed in order to alert practitioners and parents of the susceptibility to deficiencies in the Vitamin B Complex or cerebral folate.

### Prevention Methods

While many of the uses of folate are generally well known, new conditions have been discovered that require the use of folates. It is well known in the art that folate should be used for nutritional supplementation of pregnant and nursing mothers. This is due to the fact that folate is essential for DNA and RNA replication and therefore it is necessary in growing and dividing cells, which are prevalent in nursing mothers and newborns. It is also known that folate, as well as vitamin B12, may be used to address neurological conditions, including depression. However, what was not known is that some thyroid-related medical conditions can lead to cerebrospinal folate deficiencies.

Therefore, it is the subject of this invention to disclose methods and compositions of administering folate and vitamin B12 to those susceptible for developing cerebrospinal folate deficiencies and therefore prevent the harmful, adverse conditions that arise from folate deficiencies.

Some of the harmful conditions that arise from cerebrospinal folate deficiencies affect development of fetuses and newborns. However, developmental problems are not limited to fetuses and newborns, as older children, adolescents, young adults and adults can be affected as well. Some of the first symptoms associated with cerebrospinal folate deficiencies are lower IQs and cognitive dysfunction. As the condition progresses, developmental delay, psychomotor regression, seizures, mental retardation, autistic features, behavioral issues and social problems may present themselves. As conditions worsen, physical function is impaired. These are only a few of the conditions that may arise from cerebrospinal folate deficiency brought upon through thyroid-related medical conditions. The methods and compositions discussed herein will prevent and have been shown to alleviate and help correct these symptoms.

One embodiment of this invention provides a method to prevent harmful conditions that arise from thyroid-related medical conditions. This embodiment comprises administering folate and vitamin B12 to people suffering from such thyroid-related medical conditions.

Administration of the folate and vitamin B12 can be done in any manner already known in the art. In a preferred embodiment, this invention provides a method to prevent and/or treat harmful conditions that arise from hypothyroidism. Hypothyroidism results in decreased thyroid function and decreased hormone production, which regulates the endocrine system. It has been recently and surprisingly found that hypothyroidism can lead to cerebrospinal folate deficiency and all of the problems that arise from decreased folate levels. What is of even greater concern is that many of these patients suffering with cerebrospinal folate deficiency are infants whose nervous system is still developing and lack folate at a crucial point in their development. In some cases, the damage cannot be completely undone. Since the folate is deficient at such a crucial moment in development, the adverse conditions can be severe. One embodiment of this invention is to administer folate and vitamin B12 to people with hypothyroidism. This administration of folate and vitamin B12 will help to prevent problems and conditions that arise from cerebrospinal folate deficiency.

In a preferred embodiment, a reduced folate is administered with vitamin B12 to a person with thyroid-related medical conditions. A non-exclusive list of examples of reduced folates are: 10-formyltetrahydrofolic acid, 5-formyltetrahydrofolic acid, 5-forminino tetrahydrofolic acid, 5,10-methenyltetrahydrofolic acid, and 5,10-methyltetrahydrofolic acid. In a more preferred embodiment, 5-methyl tetrahydrofolic acid is administered with vitamin B12 to persons with thyroid-related medical conditions.

The amount of folate administered by the methods and compositions of this invention will depend upon the size, age, and severity of the condition of the patient. Generally the National Institutes of Health, Office of Dietary Supplements (NIH) generally recommended dosage guidelines will suffice. This is also true for the administration of vitamin B12, iron, calcium, vitamin D, and L-carnitine, as well as other vitamins of the Vitamin B Complex and the Other Elements. The amount of Vitamin B Complex and Other Elements are present in amounts that are within the USFDA recommend daily guidelines. In severe cases the amounts can be increased. Dosage amounts may need to be lower than NIH generally recommended dosage guidelines in the event of preventive measures, or in the event the patient is already taking supplements containing the foregoing, or in the event the patient is a premature infant or very newborn neonate.

In one embodiment, the amount of folate to be administered by the methods and compositions of this invention should be from about 0.5 mg to about 0.1 mg of folate per kg of weight (of the patient) per day. In other cases, higher dosages of folate at 2-3 mg/kg/day are required to normalize cerebrospinal folate levels. Yet, in other cases, where preventive measures are being taken, or when the patient is a fetus, premature newborn or term neonate, then dosage amounts can be lower than the foregoing.

In one embodiment, the amount of reduced folate to be administered by the methods and compositions of this invention should be from about 0.1 mg to about 1.0 mg of folate per kg of weight (of the patient) per day. In a preferred embodiment, the amount of reduced folate to be administered by the methods and compositions of this invention should be from about 0.5 mg to about 0.1 mg of folate per kg of weight (of the patient) per day. In other cases, higher dosages of folate at about 2-3 mg/kg/day are required to normalize cerebrospinal folate levels. Yet, in other cases were preventive measures are being taken, or

TABLE 1
Adequate Intake for Folate for Infants

| Age | Males and Females |
|---|---|
| (months) | (μg/day) |
| 0 to 6 | 65 |
| 7 to 12 | 80 |

TABLE 2
Recommended Dietary Allowances for Folate for
Children and Adults

| Age | Males and Females | Pregnancy | Lactation |
|---|---|---|---|
| (years) | (μg/day) | (μg/day) | (μg/day) |
| 1-3 | 150 | N/A | N/A |
| 4-8 | 200 | N/A | N/A |
| 9-13 | 300 | N/A | N/A |
| 14-18 | 400 | 600 | 500 |
| 19+ | 400 | 600 | 500 |

TABLE 3
Recommended Dietary Allowances (RDAs) for Vitamin B12

| Age | Male | Female | Pregnancy | Lactation |
|---|---|---|---|---|
| Birth to 6 months* | 0.4 mcg | 0.4 mcg | | |
| 7-12 months* | 0.5 mcg | 0.5 mcg | | |
| 1-3 years | 0.9 mcg | 0.9 mcg | | |
| 4-8 years | 1.2 mcg | 1.2 mcg | | |
| 9-13 years | 1.8 mcg | 1.8 mcg | | |
| 14+ years | 2.4 mcg | 2.4 mcg | 2.6 mcg | 2.8 mcg |

TABLE 4
Recommended Adequate Intake for Infants and
Recommended Dietary Allowances for Iron for Infants
(7 to 12 months), Children, and Adults

| | Males | Females | Pregnancy | Lactation |
|---|---|---|---|---|
| Age | (mg/day) | (mg/day) | (mg/day) | (mg/day) |
| Infants | 0.27 | 0.27 | N/A | N/A |
| 7 to 12 months | 11 | 11 | N/A | N/A |
| 1 to 3 years | 7 | 7 | N/A | N/A |
| 4 to 8 years | 10 | 10 | N/A | N/A |
| 9 to 13 years | 8 | 8 | N/A | N/A |
| 14 to 18 years | 11 | 15 | 27 | 10 |
| 19 to 50 years | 8 | 18 | 27 | 9 |
| 51+ years | 8 | 8 | N/A | N/A |

TABLE 5
Adequate Intake (AIs) for Calcium

| Age | Male | Female | Pregnant | Lactating |
|---|---|---|---|---|
| Birth to 6 months | 210 mg | 210 mg | | |
| 7-12 months | 270 mg | 270 mg | | |
| 1-3 years | 500 mg | 500 mg | | |
| 4-8 years | 800 mg | 800 mg | | |
| 9-13 years | 1,300 mg | 1,300 mg | | |
| 14-18 years | 1,300 mg | 1,300 mg | 1,300 mg | 1,300 mg |
| 19-50 years | 1,000 mg | 1,000 mg | 1,000 mg | 1,000 mg |
| 50+ years | 1,200 mg | 1,200 mg | | |

TABLE 6
Adequate Intakes (AIs) for Vitamin D

| Age | Children | Men | Women | Pregnancy | Lactation |
|---|---|---|---|---|---|
| Birth to 13 years | 5 mcg | | | | |
| | (200 IU) | | | | |
| 14-18 years | | 5 mcg | 5 mcg | 5 mcg | 5 mcg |
| | | (200 IU) | (200 IU) | (200 IU) | (200 IU) |
| 19-50 years | | 5 mcg | 5 mcg | 5 mcg | 5 mcg |
| | | (200 IU) | (200 IU) | (200 IU) | (200 IU) |
| 51-70 years | | 10 mcg | 10 mcg | | |
| | | (400 IU) | (400 IU) | | |
| 71+ years | | 15 mcg | 15 mcg | | |
| | | (600 IU) | (600 IU) | | |

The recommended amount of L-carnitine to be administered is from about 400 mg to about 3000 mg for adults, and from about 20 mg to about 400 mg for children. Lower amounts may be necessary in preventative cases or premature/neonate cases.

While these ranges can be used as a guide, the best practice is for the physician to determine the amount based upon the age, weight and severity of the, condition. For example: a patient (later referred to as Example 2) suffered cerebrospinal folate deficiency from birth until receiving treatment more than five years after birth. The child was treated with folinic acid at 5 mg twice per day. This dosage was necessary to address the extreme deficiency the child had developed starting in utero. In other cases, especially newborns, who may not have yet manifested any clinical presentations, lower allowances can suffice for prevention purposes.

In another example: a patient (later referred to as Example 1, and also a twin of Example 2) suffered from clinical signs of cerebrospinal folate deficiency at birth. Example 1 received infant milk formula that contained vitamin B12. However, it was not until Example 1 received a separate multivitamin nutritional supplement that contained 2 mcg of vitamin B12 (500% more than the 0.4 mcg NIH recommended daily allowance) that Example 1 showed hematological response. It is noted that the multivitamin nutritional supplement was Poly-Vi-Sol. Example 2 received a version of Poly-Vi-Sol that was fortified with iron, but that contained no vitamin B12. It is further noted that neither the version of Poly-Vi-Sol (without the iron fortification) nor the Poly-Vi-Sol (with iron fortification) contained any folate. Thus, the disclosed subject matter addresses the need for Poly-Vi-Sol, and other vitamin supplements that are given to neonates, infants and toddlers, to contain the methods and compositions described herein.

As further addressed in the Examples below, although Example 1 exhibited at birth and in the months thereafter signs of cerebrospinal folate deficiency, over time the damages Example 1 suffered as a result of cerebrospinal folate deficiency were not as severe as Example 2. This is due to Example 1 receiving additional vitamin B12 supplementation after birth and obtaining hematological response.

To the extent that this invention is treating a fetus, a premature newborn or a term neonate who may also be receiving adequate nutritional supplementation from other sources given such individual's then current medical status, trace amounts of folate and vitamin B12 can be sufficient to prevent the thyroid-related medical conditions. What is important is to determine the total amounts of these vitamins from all of the mother's nutritional intake in determining the proper amounts to be administered by this embodiment of the invention.

In another embodiment, this invention provides a method to prevent and/or treat harmful conditions that arise from hyperthyroidism. While it is more common that folate deficiencies arise from hypothyroidism, patients with hyperthyroidism are also at risk due to the fact that they are taking drugs that suppress thyroid function. The administration of folate, or reduced folates, and vitamin B12 will help prevent or treat problems in conditions that arise when the thyroid is suppressed to levels that will cause folate deficiency. One of the discoveries of this invention is that there are incidents where people who have been taking anti-thyroid drugs have taken an amount that actually lowered the thyroid function to below normal or that have adversely affected the hematological or hepatic conditions of the patient. A preferred embodiment of the invention prevents and/or treats the complications that arise from such abnormal function. This preferred embodiment would couple treatment of anti-thyroid drugs with the administration of a folate, or a reduced folate, and vitamin B12. In another embodiment, this administration can also be coupled with the administration of iron, L-carnitine, calcium or vitamin D, which can be administered by any manner already known in the art. In another embodiment, this administration can also be coupled with vitamins from the Vitamin B Complex and/or Other Elements, which can be administered by any manner already known in the art.

In one embodiment, the condition that is a result of improper thyroid function is cerebrospinal folate deficiency. In another embodiment, masked megaloblastic anemia or a masked macrocytic anemia, or a macrocytic anemia is the condition that is a result of improper thyroid function. Both of these conditions have recently been linked to improper thyroid function. The present invention presents methods and compositions to prevent and treat cerebrospinal folate deficiency and masked macrocytic anemias that have arisen in patients with improper thyroid function.

In one embodiment, a folate and vitamin B12 are administered to prevent masked megaloblastic anemia or a masked macrocytic anemia, or a macrocytic anemia in a person that suffers adverse conditions as a result of thyroid-related medical conditions. In cases of masked megaloblastic anemia or masked macrocytic anemia, or a macrocytic anemia this administration can be coupled with the administration of iron. The amount of iron necessary will be dependent upon the amount of iron anemia. It is to be cautioned, that overdoses of iron are also harmful and could interfere with certain thyroid drugs' absorption rates. In another embodiment this administration can also be coupled with the administration of calcium, yet, it should also be noted that calcium can interfere with the absorption rate of certain thyroid drugs. Both the iron and the calcium can be administered by any manner already known in the art. In another embodiment, this administration can also be coupled with the administration of L-carnitine or vitamin D, which can be administered by any manner already known in the art. In another embodiment, this administration can also be coupled with vitamins from the Vitamin B Complex and/or Other Elements, which can be administered by any manner already known in the art.

In one embodiment, the condition that is a result of a thyroid-related medical condition is hepatic dysfunction. In thyroid-related medical conditions, the hepatic dysfunction can be idiosyncratic or difficult to diagnose given its unpredictability and sudden onset. The liver is one of the major sites for folate and vitamin B12 storage and metabolism. The present invention provides methods and compositions to prevent and treat the adverse effects caused by hepatic dysfunction, by the provision of folate, or a reduced folate, and vitamin B12. In another embodiment, this administration can also be coupled with the administration of iron, L-carnitine, calcium or vitamin D, which can be administered by any manner already known in the art. In another embodiment, this administration can also be coupled with vitamins from the Vitamin B Complex and/or Other Elements, which can be administered by any manner already known in the art.

Another condition that results in improper thyroid function is hypothyroxinemia or other temporary period of hypothyroidism. Hypothyroxinemia is when a person suffers from an abnormally low concentration of thyroxine in the blood. Hypothyroxinemia has also been discovered to be linked to folate deficiency. In one embodiment of this invention, folate, or reduced folates, and vitamin B12 are administered to a person with hypothyroxinemia to prevent and/or treat complications as a result of hypothyroxinemia. In another embodiment, this administration can also be coupled with the administration of iron, L-carnitine, calcium or vitamin D, which can be administered by any manner already known in the art. In another embodiment, this administration can also be coupled with vitamins from the Vitamin B Complex and/or Other Elements, which can be administered by any manner already known in the art.

Many times when an individual is treated with radioactive iodine this impairs normal thyroid function. One embodiment of this invention prevents and/or treats complications that arise from treatment with radioactive iodine through the administration of folate, or reduced folates, and vitamin B 12. Additionally, persons can undergo surgery on or related to the thyroid gland or have other medical procedures that result in the reduced size or activity of the thyroid. Complications arising from such treatments can be alleviated by the administration of folate, or reduced folates, and vitamin B12. In another embodiment, this administration can also be coupled with the administration of iron, L-carnitine, calcium or vitamin D, which can be administered by any manner already known in the art. In another embodiment, this administration can also be coupled with vitamins from the Vitamin B Complex and/or Other Elements, which can be administered by any manner already known in the art.

A person who may be taking a thyroid stimulating drug to increase the amount of thyroid hormone can suffer conditions related to the naturally decreased amount of thyroid hormone. In a preferred embodiment of the invention, a folate, or a reduced folate, and vitamin B 12 are administered along with the thyroid stimulating drug to a person taking a thyroid stimulating drug. In another embodiment, this administration can also be coupled with the administration of iron, L-carnitine, calcium or vitamin D, which can be administered by any manner already known in the art. In another embodiment, this administration can also be coupled with vitamins from the Vitamin B Complex and/or Other Elements, which can be administered by any manner already known in the art.

Clinical conditions from abnormal thyroid function in pregnant or nursing women can be passed along to the fetus and/or later newborn. One embodiment of this invention will administer folate, or a reduced folate, and vitamin B12 to these pregnant or nursing women. Some thyroid-related medical conditions prevent absorption and/or reduction of folates in pregnant women. Thus, even though a pregnant woman can be taking a prenatal vitamin supplement that includes a folate (generally folic acid), the thyroid-related medical conditions are preventing the biologically active folates from reaching the fetus. Thus, the fetus then suffers the adverse conditions from the thyroid-related medical condition of the mother. The embodiments of this invention, providing reduced folates to a pregnant woman with thyroid-related medication conditions, will help prevent the fetus from suffering adverse effects by providing the necessary reduced folates for development. In another embodiment, this administration can also be coupled with the administration of iron, L-carnitine, calcium or vitamin D, which can be administered by any manner already known in the art. In another embodiment, this administration can also be coupled with vitamins from the Vitamin B Complex and/or Other Elements, which can be administered by any manner already known in the art.

Additionally, other thyroid-related medical conditions can cause vitamin B 12 deficiencies in pregnant women. Even if the mother is taking a prenatal vitamin with folates and/or vitamin B12, the thyroid-related medical conditions can impair the mother's ability to reduce the folates into its biologically active form. Thus, the newborn suffers adverse conditions. The embodiments of this invention provide vitamin B12 to a pregnant woman with thyroid-related medication conditions and will help prevent the fetus from adverse effects by providing the necessary vitamin B12 to enable the reduction of folates needed for development. In another embodiment, this administration can also be coupled with the administration of iron, L-carnitine, calcium or vitamin D, which can be administered by any manner already known in the art. In another embodiment, this administration can also be coupled with vitamins from the Vitamin B Complex and/or Other Elements, which can be administered by any manner already known in the art.

Common treatment for thyroid conditions is the administration of anti-thyroid drugs. An anti-thyroid drug is a hormone antagonist acting upon thyroid hormones. Examples include: propylthiouracil, methimazole, carbimazole, potassium perchlorate, and potassium iodide. Since people taking an anti-thyroid drug are susceptible to developing conditions related to decreased folate levels, one embodiment of this invention provides a composition which comprises an anti-thyroid drug coupled with a folate, or a reduced folate, and vitamin B12. Administration of these nutrients along with the drug would prevent a folate deficiency from arising or treat a folate deficiency. Propylthiouracil is a common anti-thyroid drug. Propylthiouracil is a thioamide drug used to treat hyperthyroidism (including Graves disease) by decreasing the amount of thyroid hormone produced by the thyroid gland. PTU inhibits the enzyme thyroperoxidase. Propylthiouracil is classified as Drug Class D in pregnancy. Class D signifies that there is positive evidence of human fetal risk. As of 2009, the Food and Drug Administration issued a warning with respect to Propylthiouracil use due to the adverse hepatic damage that it causes. Maternal benefit can outweigh fetal risk in life-threatening situations. The primary effect on the fetus from transplacental passage of PTU is the production of a mild hypothyroidism when the drug is used close to term. This usually resolves within a few days without treatment. The hypothyroid state can be observed as a goiter in the newborn and is the result of increased levels of fetal pituitary thyrotropin. In one embodiment, a composition of propylthiouracil, folate, or a reduced folate, and vitamin B12 is created to be administered to people who need to take anti-thyroid drugs. Methimazole is another common anti-thyroid drug. In another embodiment, a composition of methimazole, folate, or a reduced folate, and vitamin B12 is created to be administered to people who need to take anti-thyroid drugs. This invention is not limited to the specific anti-thyroid drugs that are mentioned, rather a composition of any anti-thyroid drug can be coupled with a folate, or reduced folate, and vitamin B12. In another embodiment, this administration can also be coupled with the administration of iron, L-carnitine, calcium or vitamin D, which can be administered by any manner already known in the art. In another embodiment, this administration can also be coupled with vitamins from the Vitamin B Complex and/or Other Elements, which can be administered by any manner already known in the art.

### Treatment Methods

Many embodiments of this invention require the administration of folate, or reduced folates, and vitamin B12. Folates are administered to treat the folate deficiency created by the thyroid-related medical conditions. In one embodiment, folic acid is the folate that is administered with the vitamin B12. Folic acid is not biologically active, but it is an effective treatment for many people who have the ability to convert folic acid into its tetrahydrofolate derivatives.

In some instances folic acid treatment is not enough as folic acid is not the biologically active form of folate. Some individuals have difficulty reducing folic acid into its more biologically active form, therefore, it is necessary to provide these individuals with a reduced folate. A preferred embodiment of the invention is the administration of a reduced folate with vitamin B12. It is estimated that administration of a reduced folate with vitamin B12 is sufficient to prevent and treat a large percentage of people with thyroid conditions. However, a material percentage must still receive 5-methyltetrahydrofolic acid and vitamin B12 to adequately prevent and/or treat the conditions brought upon by the folate deficiencies due to thyroid-related medical conditions. This is the most preferred embodiment of the invention. Indeed, even if an individual's blood levels of folate are treated and brought to normal, if the degree of folate deficiency was significant or prolonged over a sustained period of time such that the individual's folate stores were depleted, then the cerebrospinal folate levels will remain decreased despite normalization of folate levels in the blood. Further, while in some cases the treatment of folate can be enough to treat the folate deficiencies, in other cases the administration of vitamin B12 is essential Vitamin B12 is essential for folates to become biologically active. It has been observed that one can suffer cerebrospinal folate deficiency and yet have normal folate blood levels. That is because there is folate that is in the blood, however, because of the deficiency in vitamin B12, the folate does not become biologically active.

For example: Example 1 and Example 2 (as discussed below) were born and diagnosed with hypothyroidism. Upon birth. Example 1 presented with more severe clinical conditions than Example 2. However, Example 1 received an additional multivitamin nutritional supplement that included 2 mcg of vitamin B 12. Example 2 did not receive the same multivitamin nutritional supplement that included 2 mcg of vitamin B 12. Approximately five years after birth, Example 2 was tested for cerebrospinal folate deficiency and was found to be deficient in cerebrospinal folate. Example 1 was tested approximately four months after Example 2's cerebrospinal folate test and was normal in cerebrospinal folate, however, Example 1's cerebrospinal folate value was lower than the midpoint of the normal range for cerebrospinal folate.

### Compositions

The disclosed compositions comprise folate at an amount effective to treat the condition for which the patient is being treated. Folate is an essential water-soluble B vitamin that occurs naturally in food. As a result of these important metabolic activities, several dietary derivatives of folate are manufactured as supplements. Although most of the derivatives are capable of becoming converted into the metabolically active form (6S) 5-methyltetrahydrofolate, the enzyme kinetics of such conversion can differ dramatically as well as the absorption rate and it is these differences that are important in determining the hierarchy of performance. As such, L-methylfolate and derivatives thereof can be preferred over other reduced folates (including folinic acid) due to its enzyme kinetics and conversion benefits.

Folates are a group of pteroyglutamate acids that become structurally and functionally altered when reduced (adding electrons) or oxidized (removing electrons). In humans, folates are absorbed most readily as 5-methyltetrahydrofolate and it is the principal circulating form of folate. Other derivatives are hydrolyzed in the intestinal jejunum and the liver to the active form with an intermediate stable form (5, 10-methylenetetrahydrofolate), 5-methyltetrahydrofolate is the predominant form of folate in the circulatory system and is the type of folate that can cross the blood-brain barrier. 5-methyltetrahydrofolate is critical for brain development and normal mental health.

In the disclosed compositions, folate can be present at from about 200 mcg to about 7 mg, from about 200 mcg to about 400 mcg, from about 190 mcg to about 390 mcg, from about 210 mcg to about 410 mcg, from about 400 mcg to about 800 mcg, from about 390 mcg to about 790 mcg, from about 410 mcg to about 810 mcg, from about 1.2 mg to about 1.9 mg, from about 2.1 mg to about 2.9 mg, from about 3.1 mg to about 3.9 mg, from about 4.1 mg to about 4.9 mg, from about 5.1 mg to about 5.9 mg, or from about 6.1 mg to about 6.9 mg. Still further, the disclosed compositions can contain about 190 mcg, 200 mcg, 210 mcg, 390 mcg, 400 mcg, 410 mcg, 790 mcg, 800 mcg, 810 mcg, 990 mcg, 1 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, or 3.2 mg of folate, where any of the stated values can form an upper or lower endpoint of a range.

In the disclosed compositions, vitamin B12 can be present at from about 200 mcg to about 3 mg, from about 200 mcg to about 500 mcg, from about 190 mcg to about 490 mcg, from about 210 mcg to about 510 mcg, from about 500 mcg to about 1 mg, from about 490 mcg to about 990 mcg, from about 510 mcg to about 1.1 mg, from about 1 mg to about 1.5 mg, from about 900 mcg to about 1.4 mg, from about 1.1 mg to about 1.6 mg, from about 1.5 mg to about 2.0 mg, from about 1.4 mg to about 1.9 mg, from about 1.6 mg to about 2.1 mg, from about 2.0 mg to about 2.5 mg, from about 1.9 mg to about 2.4 mg, from about 2.1 mg to about 2.6 mg, from about 2.5 mg to about 3.0 mg, from about 2.4 mg to about 2.9 mg, or from about 2.6 mg to about 3.1 mg. Still further, the disclosed compositions can contain about 450 mcg, 500 mcg, 550 mcg, 950 mcg, 1 mg, 1.1 mg, 1.4 mg, 1.5 mg, 1.6 mg, 1.9 mg, 2.0 mg, 2.1 mg, 2.4 mg, 2.5 mg, 2.6 mg, 2.9 mg, 3.0 mg, or 3.1 mg of vitamin B12, where any of the stated values can form an upper or lower endpoint of a range.

In the disclosed compositions. L-Carnatine can be present at from about 20 mg to about 4.100 mg, from about 20 mg to about 100 mg, from about 100 mg to about 200 mg, from about 200 mg to about 300 mg, from about 300 mg to about 500 mg, from about 500 mg to about 1,000 mg, from about 1,000 mg to about 2,000 mg, from about 2,000 mg to about 3,000 mg, or from about 3,000 mg to about 4,000 mg. Still further, the disclosed compositions can contain about 90 mg, 100 mg, 110 mg, 200 mg, 190 mg, 210 mg, 300 mg, 290 mg, 310 mg, 390 mg, 400 mg, 410 mg, 600 mg, 1.100 mg, 1,600 mg, 2,100 mg, 2.600 mg, 3,100 mg, 3,600 mg, or 4,100 mg of L-carnatine, where any of the stated values can form an upper or lower endpoint of a range.

In the disclosed compositions, vitamin B6 can be present at from about 20 mg to about 62 mg. Still further, the disclosed compositions can contain about 22 mg, 27 mg, 32 mg, 37 mg, 42 mg, 47 mg, 52 mg, 57 mg, or 62 mg of vitamin B6, where any of the stated values can form an upper or lower endpoint of a range.

One embodiment of the invention includes a composition of an anti-thyroid drug, folate, and vitamin B12. In one embodiment of this invention, this composition would be administered to a pregnant woman with hyperthyroidism. The anti-thyroid drugs could be any drug that has been approved to treat an overactive thyroid gland or suppress thyroid function. A nonexclusive list includes, propylthiouracil, methimazole, carbimazole, potassium perchlorate, and potassium iodide. The amounts of anti-thyroid drug would be the amounts a physician would prescribe that is appropriate for the patient's condition. The amount of folate should be at least 30% or more of the generally recommended allowance by the NIH, depending on what additional supplements the patient may be taking. The amount of vitamin B12 should be at least 30% or more of the generally recommended allowance by the NIH, depending on what additional supplements the patient may be taking. Dosage amounts may need to be increased or decreased depending on such factors. For instance, with respect to Example 1 (as discussed above and below), 2 mcg of vitamin B 12 per day was required for Example 1 to show hematological improvement, which equates to a 500% increase over NIH's recommended daily allowance. This composition may be administered by any means necessary already known in the art. In a preferred embodiment, the composition would be administered in a capsule containing all three elements. The capsule could be made by any means necessary already known in the art.

The combination of an anti-thyroid drug and folate and vitamin B12 will serve to provide folate and vitamin B12 to the patient and prevent folate deficiencies including cerebrospinal folate deficiency. The vitamin B12 is necessary to help the folate transport into the cerebral spinal fluid.

In a more preferred embodiment of the invention, a composition would include an anti-thyroid drug, a reduced folate, and vitamin B12. The amount of reduced folate should be at least 30% or more of the generally recommended allowance of folic acid by the NIH, depending on what additional supplements the patient may be taking. Dosage amounts may need to be increased or decreased depending on such factors. Since reduced folates are more biologically active, a reduced folate would be more effective in treating folate deficiencies.

Additionally, those individuals who reduce folic acid would be benefitted by taking a reduced folate. It is estimated that this composition would be effective for a significant percentage of persons with cerebrospinal folate deficiency. For the remaining population, 5-methyltetrahydrofolic acid is necessary.

In a more preferred embodiment of the invention, a composition would include an anti-thyroid drug, 5-methyltetrahydrofolic acid, and vitamin B12. The amount of 5-methyltetrahydrofolic acid should be at least 30% or more of the generally recommended allowance for folic acid by the NIH. In another preferred embodiment, the amount of 5-methyltetrahydrofolic acid should be based on a formula of 0.1-1.0 mg/kg/day. Depending on what additional supplements the patient may be taking, dosage amounts may need to be increased or decreased depending on such factors.

Since other complications arise from thyroid-related medical conditions, another embodiment of this invention includes a composition that includes an anti-thyroid drug, a folate, vitamin B12, and/or iron, and/or L-carnitine and/or calcium and/or vitamin D. L-carnitine has shown to improve mental development in cellular metabolism. These functions are necessary for those susceptible to folate deficiencies. In addition, Example 2 (described below) became hypothyroid as a result of anti-thyroid drug treatment in the mother. At the time Example 2 was diagnosed with cerebrospinal folate deficiency, Example 2 also had a deficiency in L-carnitine. Anti-thyroid drugs have been shown to cause hypothyroidism, and hypothyroidism causes iron deficiencies; therefore, iron supplements can be suitable to correct any iron deficiency. Further, to the extent the hyperthyroidism treatment causes hypothyroidism, hypothyroidism has been found to be associated with hypoparathyroidism. Calcium is effective in the treatment of hypoparathyroidism, and vitamin D assists in the absorption of calcium. In other embodiments of this invention, other vitamins of the Vitamin B Complex and/or the Other Elements would be utilized.

In another embodiment of the invention, a composition would include a thyroid-stimulating drug, folate, and vitamin B12. In one embodiment of this invention, this composition would be administered to an individual with hypothyroidism. The thyroid-stimulating drug could be any drug or hormone that has been approved to treat underactive thyroid function or that is a natural thyroid replacement therapy such as dessicated thyroid hormone. A nonexclusive list includes. Levothyroxine. Levothyroxine Sodium, Liothyronine Sodium, Liotrix, Thyroglobulin, Thyroid, Thyroxine. Triiodothyronine, Levoxyl, Synthroid, Levo-T, Unithroid, Levothroid, Levoxine, Levolet, Novothyrox, Triostat, Cytomel and Thyrolar. The amounts of thyroid-stimulating drug would be the amounts a physician would prescribe that is appropriate for the patient's condition. The amount of folate should be at least 30% or more of the generally recommended allowance by the NIH, depending on what additional supplements the patient can be taking. The amount of vitamin B12 should be at least 30% or more of the generally recommended allowance by the NIH, depending on what additional supplements the patient can be taking. Dosage amounts can need to be increased or decreased depending on such factors. For instance, with respect to Example 1 (as discussed above and below), 2 mcg of vitamin B12 per day was required for Example 1 to show hematological improvement, which equates to a 500/o increase over NIH's recommended daily allowance. This composition can be administered by any means necessary already known in the art. In a preferred embodiment, the composition would be administered in a capsule containing all three elements. The capsule could be made by any means necessary already known in the art.

The combination of a thyroid-stimulating drug and folate and vitamin B12 will serve to provide folate and vitamin B12 to the patient and prevent folate deficiencies including cerebrospinal folate deficiency. The vitamin B12 is necessary to help the folate transport into the cerebral spinal fluid. In a more preferred embodiment of the invention, a composition would include a thyroid-stimulating drug, a reduced folate, and vitamin B12. The amount of reduced folate should be at least 30% or more of the generally recommended allowance of folic acid by the NIH, depending on what additional supplements the patient can be taking. Dosage amounts can need to be increased or decreased depending on such factors. Since reduced folates are more biologically active, a reduced folate would be more effective in treating folate deficiencies. Additionally, those individuals who reduce folic acid would be benefitted by taking a reduced folate. It is estimated that this composition would be effective for a significant percentage of persons with cerebrospinal folate deficiency. For the remaining population, 5-methyltetrahydrofolic acid is necessary.

In a more preferred embodiment of the invention, a composition would include a thyroid-stimulating drug, 5-methyltetrahydrofolic acid, and vitamin B12. The amount of 5-methyltetrahydrofolic acid should be at least 30% or more of the generally recommended allowance for folic acid by the NIH. In another embodiment, the amount of 5-methyltetrahydrofolic acid should be based on a formula of about 0.1-1.0 mg/kg/day. Depending on what additional supplements the patient can be taking, dosage amounts can need to be increased or decreased depending on such factors.

Since other complications arise from thyroid-related medical conditions, another embodiment of this invention includes a composition that includes a thyroid stimulating drug, a folate, vitamin B12, and/or iron, and/or L-carnitine and/or calcium and/or vitamin D. L-carnitine has shown to improve mental development in cellular metabolism. These functions are necessary for those susceptible to folate deficiencies. In addition, Example 2 (described below) became hypothyroid as a result of anti-thyroid drug treatment in the mother. At the time Example 2 was diagnosed with cerebrospinal folate deficiency, Example 2 also had a deficiency in L-carnitine. Hypothyroidism causes iron deficiencies; therefore, iron supplements can be suitable to correct any iron deficiency. Further, hypothyroidism has been found to be associated with hypoparathyroidism. Calcium is effective in the treatment of hypoparathyroidism, and vitamin D assists in the absorption of calcium. In other embodiments of this invention, other vitamins of the Vitamin B Complex and/or the Other Elements are utilized.

### Delivery Methods

In vivo application of the disclosed compositions can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compositions disclosed herein can also be administered utilizing liposome technology, controlled release capsules, tablets, pills, and implants, implantable pumps, and biodegradable containers. These delivery methods can, advantageously, provide a uniform dosage over an extended period of time. There is a need to have a controlled release composition when using folate in combination with calcium in patients taking thyroid drug such that the calcium is released 4-6 hrs after the thyroid drug is released. In a combined pill that first releases the thyroid drug consistent with its normal absorption profile and then 4-6 hrs. later, the calcium and folate is released. Calcium can interfere with the absorption of thyroid hormone. Thus co-administration of calcium with a thyroid drug, as detailed herein, should involve the controlled release of the calcium so that it is absorbed after the thyroid drug.

The compounds can also be administered in their salt derivative forms or crystalline forms.

The compositions disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question

Therapeutic application of the disclosed compositions can be accomplished by any suitable therapeutic method and technique presently or prospectively known to those skilled in the art. Further, compositions disclosed herein have use as starting materials or intermediates for the preparation of other useful compounds and compositions.

Compositions disclosed herein can be locally administered at one or more anatomical sites, injected or topically applied, optionally in combination with a pharmaceutically acceptable carrier such as an inert diluent. Compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject as skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

### EXAMPLES

The following examples illustrate the medical conditions presented in twins who were born to a mother diagnosed with hyperthyroidism who had excessive anti-thyroid drug treatment during the pregnancy that, as a result, created a hypothyroid state in the mother as well as a hypothyroid state in the twin neonates.

Example 1 and Example 2 were both infant patients. Both Example 1 and Example 2 were born to a mother who was diagnosed with hyperthyroidism and was treated with excessive antithyroid drugs during the pregnancy, thus creating a hypothyroid state in the mother, and in the fetuses. The mother also developed diabetes mellitus during the pregnancy. It was later determined that both Example 1 and Example 2 were hypothyroid in utero.

Both Examples 1 and 2 received thyroid stimulating drugs after birth and became euthyroid within approximately one week of birth. Immediately after birth, Example 1 had evidence of megaloblastic anemia and neutropenia. Example 2 had evidence of masked megaloblastic anemia, as well as neutropenia that can have been masked. It is notable that Example 2's hematological testing was performed approximately one hour after Example 1's hematological testing, a period of time in which neutrophil and white blood cell values have been shown to rise. Both Example 1 and Example 2 showed signs of hepatic dysfunction. It is notable that the mother showed signs of idiosyncratic hepatic dysfunction during the pregnancy while taking anti-thyroid drugs.

Both Example 1 and Example 2 were treated for iron deficiencies with iron supplements. Example 1 and Example 2 received different nutritional supplementation with respect to vitamin B 12. Although Example 2 did receive the same infant milk formula that Example 1 received, which infant milk formula contained vitamin B 12, Example 2 received less of the infant milk formula than Example 1, and Example 2 received in lieu of the infant milk formula more of the breast milk from the hypothyroid mother. Example 1 also received an additional multivitamin nutritional supplement that included 2 mcg of vitamin B12 in the form of Poly-Vi-Sol. Example 2 received a different version of the multivitamin nutritional supplement that did not include vitamin B12 in the form of Poly-Vi-Sol fortified with iron.

When Example 1 received the additional nutritional supplement containing 2 mcg of vitamin B 12, Example 1 showed prompt hematological response by an increase in reticulocytes, moving from below normal to normal, which is evidence of a treated vitamin B12 and/or folate deficiency. Example 2, however, showed regression in reticulocyte values and remained below normal, evidencing a continued vitamin B12 and/or folate deficiency.

Both Example 1 and Example 2 exhibited signs associated with cerebrospinal folate deficiency at birth and within the ensuing year, including, but not limited to, failure to thrive, drowsiness, pallor, glossitis, sepsis and septicemia, as well as neurological manifestations including cognitive impairment, movement disorders and peripheral neuropathy. For the most part, Example 2 exhibited more drastic versions of the symptoms, including behavioral and social issues and painful movement disorders.

In summary, it has been determined that the proper maternal folate metabolism, which was altered by the excessive anti-thyroid drug treatment, the mother's hypothyroidism, and pernicious anemia, critically affected delivery of folate to the embryo and transport of intact folate across the placenta. This means that Example 1 and Example 2 began to suffer from systemic folate deficiency in the womb, and systemic folate deficiency leads to cerebrospinal folate deficiency. Example 1's and Example 2's folate condition was also impacted by their own hypothyroidism and placental transfer of the mother's anti-thyroid drug. In fact, since Example 1's and 2's hypothyroidism resolved in approximately one week of birth, Example 1 and 2 experienced hypothyroxinemia. It is notable that the thyroid stimulating drugs that Example 1 and Example 2 received immediately after birth and which brought each of them to a euthyroid state within approximately a week did not sufficiently address cerebrospinal folate deficiencies, nor was the prompt hematological response seen in Example 1 after additional vitamin B12 supplementation associated with the thyroid stimulating drugs treatment.

Both Example 1 and Example 2 displayed a number of conditions consistent with cerebrospinal folate deficiency. Example 1 and Example 2 are similar in that both Example 1 and Example 2 had a mother treated with an anti-thyroid drug and that was diagnosed with hypothyroidism, thereby resulting in hypothyroidism in Example 1 and Example 2. Additionally, both Example 1 and Example 2 had goiters at birth, had similar lab treatment in the hospital after birth, and lived a somewhat similar life (food, upbringing, school, same medications and vitamin supplements, vaccinations, etc.) after discharge from the hospital. One significant difference was that Example 1 received more vitamin B12 supplementation than Example 2, and Example 1 showed prompt hematological response.

Although Example 1 suffered and continues to suffer from symptoms associated with the onset of cerebrospinal folate deficiency. Example 1's manifestations have been to a lesser degree than Example 2. Example 2 has suffered, and continues to suffer, from symptoms of cerebrospinal folate deficiency to a greater degree than Example 1. Approximately five years and three months after birth, cerebrospinal folate levels were observed for the first time in Example 2. Example 1 was tested for cerebrospinal folate deficiency approximately four months after Example 2's testing. Example 1 showed normal levels of cerebrospinal folate, which is consistent with the additional vitamin B12 support Example 1 received after birth (and the resulting hematological response), and the lesser degree of symptoms associated with the onset of cerebrospinal folate deficiency that Example 1 has suffered from. Studies have shown that the earlier the anemias associated with cerebrospinal folate deficiency are addressed, the better the adversely impacted individual can overcome more long-term effects of the associated folate deficiency. However, it is notable that although Example 1's cerebral folate value was within the normal range at testing, Example 1's cerebral folate value was below the midpoint of the normal range.

Notwithstanding Example 1's cerebrospinal folate deficiency test results, Example 1 still has permanent neurological damage resulting from cerebrospinal folate deficiency at birth, demonstrating the need for the methods and compositions of this invention. Example 2 showed below normal levels of cerebrospinal folate, which is consistent with Example 2's lack of hematological response after birth given Example 2's lesser vitamin B12 supplementation, and the higher degree of symptoms associated with cerebrospinal folate deficiency that Example 2 has suffered from. After Example 2's diagnosis, Example 2 was placed on 5-methyltetrahydrofolate in the form of folinic acid (5 mg twice per day). Within approximately four months. Example 2's cerebrospinal folate levels rose from 32 L (preferred range 40-128) to approximately 88 (above the midpoint of the range). Thus after four months of treatment, Example 2 achieved normal cerebrospinal folate levels, while Example 2 could not achieve such normal levels within the first five years of Example 2's life even when receiving multivitamins with folic acid and vitamin B12. Thus, reduced folates are critical. After 5-methyltetrahydrofolate treatment, Example 2 showed improvement in physical, behavioral and social skills.

### SPECIFIC EMBODIMENTS

Disclosed is a method of treating an individual undergoing radioactive iodine therapy that comprises administering to the individual a reduced folate. Disclosed is a method of preventing or treating decreased folate in cerebrospinal fluid that comprises selecting a neonate or infant with, or at risk of, hypothyroidism and decreased folate in cerebrospinal fluid; and administering a composition comprising folate to the neonate or infant. In another example, the folate is a reduced folate. In another example, the folate is administered at a dosage of from about 0.5 to about 0.1 mg/kg day of the neonate or infant. In another example, the administration of vitamin B12 to the neonate or infant. In another example, the neonate or infant has a masked megaloblastic anemia. In another example, the neonate or infant has a masked macrocytic anemia. In another example, the neonate or infant has a macrocytic anemia. In another example, the neonate or infant has hypothyroxinemia or temporary period of hypothyroidism. In another example, the neonate or infant has or had radioactive iodine or radiation that affects the thyroid gland, or has or had surgery on the thyroid gland. In another example, the neonate or infant takes or has taken an anti-thyroid drug or thyroid stimulating drug, or undergoes or has undergone treatment that increases or decreases thyroid hormone or thyroid function. In another example, the neonate or infant that is selected by identifying a MTHFR polymorphism in the neonate or infant. In another example, selecting the neonate or infant comprises identifying a neonate or infant whose mother has or had radioactive iodine or radiation that affects the thyroid gland or has or had surgery on the thyroid gland. In another example, selecting the neonate or infant comprises identifying a neonate or infant whose mother has or had taken an anti-thyroid drug or thyroid stimulating drug, or has or had undergone treatment that increases or decreases thyroid hormone or thyroid function. In another example, selecting the neonate or infant comprises identifying a neonate or infant whose mother has, or is at risk of, hypothyroidism. In another example, the administration of a thyroid stimulating drug or anti-thyroid drug to the neonate or infant. In another example, the administration of iron to the neonate or infant. In another example, the administration of L-carnitine to the neonate or infant. In another example, the administration of calcium or vitamin D to the neonate or infant. In another example, the reduced folate is administered with an anti-thyroid drug or thyroid stimulating drug. In another example, the administration of one or more of the following: vitamin B12, iron, L-carnitine, calcium, or vitamin D. In another example, the reduced folate is in a composition comprising either an anti-thyroid drug or thyroid stimulating drug, and one or more of the following: vitamin B12, iron, L-carnitine, calcium or vitamin D. In another example, testing the level of folate in the cerebrospinal fluid of the neonate or infant.

Disclosed is a method of preventing or treating decreased folate in cerebrospinal fluid, comprising: selecting an individual with, or at risk of, hypothyroidism; and administering a composition comprising an anti-thyroid drug or a thyroid stimulating drug and a reduced folate to the individual. In another example, the method can further comprise the administration of vitamin B12 to the individual. In another example, the individual has a masked megaloblastic anemia. In another example, the individual has a masked macrocytic anemia. In another example, the individual has a macrocytic anemia. In another example, the individual has hypothyroxinemia or period of temporary hypothyroidism. In another example, the individual has the MTHFR poly morphism. In another example, the individual has or had radioactive iodine or radiation that affects the thyroid gland, or has or had surgery on the thyroid gland. In another example, the individual takes or has taken an anti-thyroid drug or thyroid stimulating drug, or undergoes or has undergone treatment that increases or decreases thyroid hormone or thyroid function. In another example, the administration of iron to the individual. In another example, the administration of L-carnitine to the individual. In another example, the administration of calcium or vitamin D to the individual. In another example, the composition further comprises one or more of the following: vitamin B12, iron, L-carnitine, calcium, or vitamin D.

Disclosed is a method of preventing or treating decreased folate in cerebrospinal fluid, comprising selecting an individual taking an anti-thyroid drug or a thyroid stimulating drug, and having normal levels of thyroid hormone, wherein the individual has symptoms of hypothyroidism; and administering a composition comprising a reduced folate to the individual. In another example, the administration of vitamin B12 to the individual. In another example, the individual has a masked megaloblastic anemia. In another example, the individual has a masked macrocytic anemia. In another example, the individual has a macrocytic anemia. In another example, the individual has hypothyroxinemia or period of temporary hypothyroidism. In another example, the individual has the MTHFR polymorphism. In another example, the individual has or had radioactive iodine or radiation that affects the thyroid gland, or has or had surgery on the thyroid gland. In another example, the individual takes or has taken an anti-thyroid drug or thyroid stimulating drug, or undergoes or has undergone treatment that increases or decreases thyroid hormone or thyroid function. In another example, the administration of iron to the individual. In another example, the administration of L-carnitine to the individual. In another example, the administration of calcium or vitamin D to the individual. In another example, the composition further comprises one or more of the following: vitamin B12, iron, L-carnitine, calcium, or vitamin D. In another example, the reduced folate is administered with an anti-thyroid drug or thyroid stimulating drug. In another example, the administration of one or more of the following: vitamin B12, iron, L-carnitine, calcium, or vitamin D. In another example, the reduced folate is in a composition comprising either an anti-thyroid drug or thyroid stimulating drug, and one or more of the following: vitamin B12, iron, L-carnitine, calcium or vitamin D. In another example, testing the level of folate in the cerebrospinal fluid of the individual.

Disclosed is a composition comprising either an anti-thyroid drug or a thyroid stimulating drug, a reduced folate, and vitamin B12. In another example, the anti-thyroid drug is selected from the group consisting of propylthiouracil, methimazole, carbimazole and potassium perchlorate. In another example, the thyroid stimulating drug is selected from the group consisting of levothyroxine, levothyroxine sodium, liothyronine sodium, liotrix, thyroglobulin, thyroid, thyroxine, triiodothyronine, levoxyl, synthroid, levo-T, unithroid, levothroid, levoxine, levolet, novothyrox, triostat, cytomel and thyrolar. In another example, further comprising L-carnitine. In another example, further comprising calcium, or vitamin D.

Disclosed is a method of preventing or treating decreased folate in cerebrospinal fluid, comprising selecting an individual taking an anti-thyroid drug or a thyroid stimulating drug, and having normal levels of thyroid hormone, wherein the individual has symptoms of hypothyroidism, and administering a composition comprising a reduced folate to the individual.

Disclosed is a method of preventing or treating decreased folate in cerebrospinal fluid, comprising selecting an individual with, or at risk of, hypothyroidism, and administering a composition comprising an anti-thyroid drug or a thyroid stimulating drug and a reduced folate to the individual.

Disclosed is a method of preventing or treating decreased folate in cerebrospinal fluid, comprising selecting a neonate or infant with, or at risk of, hypothyroidism and decreased folate in cerebrospinal fluid, and administering a composition comprising folate to the neonate or infant.

Disclosed is a method of preventing or treating decreased folate in cerebrospinal fluid, comprising selecting an individual for whom an anti-thyroid drug or thyroid stimulating drug is indicated, and administering a composition comprising the anti-thyroid drug or thyroid stimulating drug and a reduced folate to the individual.

What is claimed is:

1. A method of treating an individual undergoing radioactive iodine therapy, comprising administering to the individual a reduced folate.

2. A method of preventing or treating decreased folate in cerebrospinal fluid, comprising:
a. selecting a neonate or infant with, or at risk of, hypothyroidism and decreased folate in cerebrospinal fluid; and
b. administering a composition comprising folate to the neonate or infant.

3. The method of claim 1, wherein the folate is a reduced folate.

4. The method of claim 1, wherein the folate is administered at a dosage of from about 0.5 to about 0.1 mg/kg day of the neonate or infant.

5. The method of claim 1, further comprising the administration of vitamin B12 to the neonate or infant.

6. The method of claim 1, wherein the neonate or infant has a masked megaloblastic anemia.

7. The method of claim 1, wherein the neonate or infant has a masked macrocytic anemia.

8. The method of claim 1, wherein the neonate or infant has a macrocytic anemia.

9. The method of claim 1, wherein the neonate or infant has hypothyroxinemia or temporary period of hypothyroidism.

10. The method of claim 1, wherein the neonate or infant has or had radioactive iodine or radiation that affects the thyroid gland, or has or had surgery on the thyroid gland.

11. The method of claim 1, wherein the neonate or infant takes or has taken an anti-thyroid drug or thyroid stimulating drug, or undergoes or has undergone treatment that increases or decreases thyroid hormone or thyroid function.

12. The method of claim 1, wherein selecting the neonate or infant comprises identifying a MTHFR polymorphism in the neonate or infant.

13. The method of claim 1, wherein selecting the neonate or infant comprises identifying a neonate or infant whose mother has or had radioactive iodine or radiation that affects the thyroid gland or has or had surgery on the thyroid gland.

14. The method of claim 1, wherein selecting the neonate or infant comprises identifying a neonate or infant whose mother has or had taken an anti-thyroid drug or thyroid stimulating drug, or has or had undergone treatment that increases or decreases thyroid hormone or thyroid function.

15. The method of claim 1, wherein selecting the neonate or infant comprises identifying a neonate or infant whose mother has, or is at risk of, hypothyroidism.

16. The method of claim 1, further comprising the administration of a thyroid stimulating drug or anti-thyroid drug to the neonate or infant.

17. The method of claim 1, further comprising the administration of iron to the neonate or infant.

18. The method of claim 1, further comprising the administration of L-carnitine to the neonate or infant.

19. The method of claim 1, further comprising the administration of calcium or vitamin D to the neonate or infant.

20. The method of claim 2, wherein the reduced folate is administered with an anti-thyroid drug or thyroid stimulating drug.

21. The method of claim 19, further comprising the administration of one or more of the following: vitamin B12, iron, L-carnitine, calcium, or vitamin D.

22. The method of claim 2, wherein the reduced folate is in a composition comprising either an anti-thyroid drug or thyroid stimulating drug, and one or more of the following: vitamin B12, iron, L-carnitine, calcium or vitamin D.

23. The method of claim 1, further comprising testing the level of folate in the cerebrospinal fluid of the neonate or infant.
